(19) United States
(12) Patent Application Publication
(10) Pub. No.: US 2019/0029728 A1
(43) Pub. Date: Jan. 31, 2019

(54) ORTHOPAEDIC STABILISATION DEVICE

(71) Applicant: CURTIN UNIVERSITY OF TECHNOLOGY, Bentley, WA (US)

(72) Inventors: Matthew Peter OLDAKOWSKI, Bentley (AU); lntan Camellia Watono OLDAKOWSKA, Bentley (AU); Philip Hobson HARDCASTLE, Bentley (AU); Thomas Brett KIRK, Bentley (AU)

(73) Assignee: CURTIN UNIVERSITY OF TECHNOLOGY, Bentley (AU)

(21) Appl. No.: 16/152,651

(22) Filed: Oct. 5, 2018

Related U.S. Application Data
(63) Continuation of application No. 14/423,077, filed on Feb. 20, 2015, now abandoned.

(30) Foreign Application Priority Data
Aug. 23, 2012 (AU) 2012903649

(51) Int. Cl. A61B 17/70; A61B 17/80; A61B 17/064; A61B 17/84; A61B 17/86; F16B 19/10

(52) U.S. Cl. CPC A61B 17/7007; A61B 17/8023; A61B 17/701; A61B 17/7014; A61B 17/7026; F16B 19/1081; A61B 17/0642; A61B 17/844; A61B 2017/8655; F16B 19/1045; A61B 17/7028

(57) ABSTRACT

An orthopaedic stabilisation device is described. The orthopaedic stabilisation device comprises a stabilisation member and at least two legs coupled to the stabilisation member. Each leg is arranged for positioning in a respective bore hole in bone, and for receiving an element within the leg such that the element can facilitate fastening the leg within the bore hole in bone. The stabilisation member may be arranged such that a length of the stabilisation member is alterable by a predefined amount, the predefined amount being adjustable.

### CROSS-REFERENCE TO RELATED APPLICATIONS

The present application claims the benefit and priority under 35 U.S.C. § 120 as a continuation of U.S. patent application Ser. No. 14/423,077, entitled “An Orthopaedic Stabilisation Device,” filed on Feb. 20, 2015, which is a U.S. National Stage of International Application No. PCT/AU2013/000943 filed on Aug. 23, 2013, which claims priority to Australian Patent Application No. 2012903649 filed on Aug. 23, 2012, the disclosures of all of which are incorporated herein by reference.

### FIELD OF THE INVENTION

The present invention relates to an orthopaedic stabilisation device.

### BACKGROUND OF THE INVENTION

The spine can become unstable due to injury, either degenerative or traumatic, or due to decompression caused by surgery for radiculopathy. It is desirable to stabilise such a spine to prevent abnormal or excessive spinal motion which can cause pain and damage to the neural system, intervertebral discs, facet joints and surrounding soft tissues.

Pain may also occur within the normal range of motion of a patient due to pathological compression of nerve roots (radiculopathy) and so in these cases stabilisation should further restrict the range of motion to the pain-free zone.

Typically, spinal fusion with a bone graft has been used to statically stabilise the spine. Fusion-inducing surgery completely immobilises the joint, preventing any segmental motion so that fusion can occur, typically 6 to 12 months after surgery.

Stabilisation of the spine typically involves implanting hardware, for example plates or rods, to span the affected vertebrae. Implantation of plates and rods can present difficulties as the plates and rods are separate from the means that are used to fasten them in place, such as fixation screws. For example, plates are typically arranged in position, and then fixation screws are inserted through apertures in each plate for fixing in place. This can result in misalignment or other difficulties during implantation. This is exacerbated by the extremely small dimensions of stabilisation hardware used for the cervical spine for which the device is primarily designed for.

### SUMMARY OF THE INVENTION

In accordance with a first aspect of the present invention, there is provided an orthopaedic stabilisation device comprising:

a stabilisation member; and
at least two legs coupled to the stabilisation member, each leg being arranged for positioning in a respective bore hole in bone and for receiving an element within the leg such that the element can facilitate fastening the leg within the bore hole in bone;
wherein the legs may be coupled to the stabilisation member prior to positioning the legs in the respective bore holes.

Providing an arrangement whereby the legs are coupled to the stabilisation member prior to positioning the legs in the respective bore holes can facilitate positioning the orthopaedic stabilisation device in a stabilising position as a single unit.

It will be appreciated that the term ‘single unit’, as used herein, refers to a device that may comprise different components, or that may be integrally formed.

In accordance with a second aspect of the present invention, there is provided an orthopaedic stabilisation device comprising:
a stabilisation member arranged to be fastenable to bones or bone portions that are to be stabilised, the stabilisation member further being arranged such that a length of the stabilisation member is alterable by a predefined amount, the predefined amount being adjustable.

In an embodiment of the second aspect, the orthopaedic stabilisation device comprises at least two legs coupled to the stabilisation member, each leg being arranged for positioning in a respective bore hole in bone and for receiving an element within the leg such that the element can facilitate fastening the leg within the bore hole in bone.

For the orthopaedic stabilisation device of the second aspect, the legs may be coupled to the stabilisation member prior to positioning the legs in the respective bore holes. Such an arrangement can facilitate positioning the orthopaedic stabilisation device in a stabilising position as a single unit.

Features described below relate to the first aspect and/or the second aspect for embodiments wherein the at least two legs are coupled to the stabilisation member.

The element that results in fastening may be a fastening element, or may cause the leg to function as a fastening element.

In one embodiment, at least one leg is moveable from a contracted configuration to an expanded configuration, the at least one leg being arranged to be received within its respective bore hole when in the contracted configuration and to be moveable to the expanded configuration when located in the bore hole for facilitating fastening the at least one leg within its respective bore hole.

In one embodiment, the at least one leg is moveable between the contracted configuration and the expanded configuration.

At least a portion of the at least one leg may be integral to the stabilisation member.

The element that results in fastening may be an actuating member, and at least one leg may be arranged to receive the actuating member such that interaction between the actuating member and the at least one leg facilitates moving the leg from the contracted configuration to the expanded configuration. For embodiments wherein the at least one leg is moveable between the contracted and expanded configurations, the at least one leg may be arranged to receive an actuating member such that interaction between the actuating member and the leg facilitates moving the leg between the contracted and expanded configurations.

In one embodiment, the at least one leg comprises a passage that is arranged to receive the actuating member.

The expanded configuration of the at least one leg may be a configuration wherein at least a portion of the at least one leg has an increased radial dimension compared to when the leg is in the contracted configuration.

The leg may be arranged to move from the contracted configuration to the expanded configuration in response to movement of the actuating member in a direction that is from the stabilisation member and towards an end of the leg that is remote from the stabilisation member.

Alternatively, the leg may be arranged to move from the contracted configuration to the expanded configuration in response to movement of the actuating member in a direction that is from an end of the leg that is remote from the stabilisation member and towards the stabilisation member.

The leg may be arranged so as to move from the contracted configuration to the expanded configuration in response to rotational movement of the actuating member, or in response to substantially linear movement of the actuating member.

In one embodiment, the legs are substantially parallel to one another. An orientation of at least one leg relative to the stabilisation member may be changed. In one embodiment, the at least one leg may be set to a desired orientation relative to the stabilisation member.

At least one leg may comprise a barb.

The stabilisation device may comprise the actuating member. At least a portion of the actuating member may be separable from the actuating member. For embodiments wherein the leg is arranged to move from the contracted configuration to the expanded configuration in response to movement of the actuating member that is in a direction from an end of the leg that is remote from the stabilisation member and towards the stabilisation member, the portion of the actuating member that is separable may be a portion of the actuating member that would otherwise at least partially protrude from the passage of the leg that receives the actuating member when the leg is in the expanded configuration.

The actuating member, or the leg, may comprise a portion that is arranged to facilitate forming a bore hole in bone.

The stabilisation device may be arranged such that each leg is moveable from a contracted to the expanded configuration, and wherein each leg can be moved from the contracted to the expanded configuration at substantially the same time. In one embodiment, the actuating member is arranged so as to effect movement of each leg from the contracted to the expanded configuration at substantially the same time.

At least one leg may comprise a plurality of expandable portions, each portion being arranged to move from a contracted configuration to an expanded configuration as the leg moves from the contracted configuration to the expanded configuration.

At least one leg may be arranged to move from the contracted to the expanded configuration by a buckling action. At least a portion of the at least one leg may comprise a notch at a predefined position along a length of the leg so as to facilitate buckling of the leg at the predefined position.

The at least one leg may comprise a plurality of longitudinally connectable portions, each connectable portion being arranged to move from a contracted configuration to an expanded configuration.

The at least one leg may be arranged such that a substantially flat portion is formed when the leg moves from the contracted to the expanded configuration, the substantially flat portion having an external surface that is substantially parallel to an axis of the leg.

At least one leg may be arranged to move from the contracted to the expanded configuration by a cantilever action. In one embodiment, the at least one leg is arranged to move from the contracted to the expanded configuration in response to the actuating member moving in a direction that is away from an end of the leg that is remote from the stabilisation member and towards the stabilisation member, and the at least one leg is arranged to retain the actuating member in a position that has caused the at least one leg to move to the expanded configuration. An internal wall of the at least one leg may be shaped so as to engage with at least a portion of the actuating member to retain the actuating member when the actuating member has caused the at least one leg to move to the expanded configuration.

At least a portion of at least one leg may be arranged to expand in a radial direction when the at least one leg interacts with the actuating member. The at least one leg may be arranged to retain the actuating member when the actuating member has interacted with the at least one leg. An internal wall of the at least one leg may be shaped so as to engage with at least a portion of the actuating member to retain the actuating member when the actuating member has been received by the at least one leg.

At least one leg may be arranged to move from the contracted to the expanded configuration such that a first portion of the at least one leg undergoes a buckling action, and a second portion of the at least one leg undergoes a cantilever action. The first portion of the at least one leg may be adjacent the stabilisation member.

The orthopaedic stabilisation device may be arranged such that a length of the orthopaedic stabilisation device is alterable.

In one embodiment, the stabilisation member is arranged such that a length of the stabilisation member is alterable.

The stabilisation member may comprise first and second stabilisation portions, the first and second stabilisation portions being arranged so as to be moveable with respect to one another.

The first and the second stabilisation portions may be at least partially coupled by at least one coupling member that is compressible and/or expandable.

In one embodiment, the at least one coupling member is a spring. The spring may be a live spring. Alternatively, the spring may be a coiled spring. In one embodiment, the at least one coupling member is a compressible element.

For embodiments wherein the length of the orthopaedic stabilisation device is alterable, the orthopaedic stabilisation device may be arranged such that the length of the orthopaedic stabilisation device is alterable by a predefined amount.

In one example, the predefined amount by which the length of the orthopaedic stabilisation device can be altered is adjustable.

In one embodiment, the first stabilisation portion is arranged to receive a pin, and the second stabilisation portion comprises an elongate slot having first and second ends, the elongate slot being arranged to receive the pin and to constrain movement of the stabilisation member to movement corresponding to the pin moving between the first and second ends of the elongate slot. A position of the pin may be alterable. In one embodiment, a position of the pin is alterable between at least two predefined positions. A plurality of pins may be positioned relative to the elongate slot so as to facilitate a plurality of movement configurations.

In an alternative embodiment, the first stabilisation portion comprises a plate portion, and the second stabilisation portion comprises an elongate slot having first and second ends, the elongate slot being arranged to receive the plate portion and to constrain movement of the stabilisation member to movement corresponding to the plate portion moving between the first and second ends of the elongate slot. The first and the second stabilisation portions may be arranged so as to provide only relative movement in a direction that is substantially parallel to a length of the stabilisation member. A length of the plate portion may be alterable. In one embodiment, the length of the plate portion is alterable between at least two predefined lengths.

For embodiments wherein the length of the orthopaedic stabilisation device is alterable, the orthopaedic stabilisation device may be arranged such that the length of the orthopaedic stabilisation device is alterable along at least one predefined direction.

It will be appreciated that the predefined direction may be along a curved path.

In one embodiment, a distance between at least two legs can be varied.

The distance between the at least two legs may be set to at least two predefined amounts, or the distance between the at least two legs can be set to any distance between two predefined distances.

The stabilisation member may be arranged to be releasably engagable with a further stabilisation member.

The stabilisation device may comprise first and second stabilisation members that are coupled together. In one embodiment, the stabilisation device comprises at least three legs wherein a first and a second leg are associated with the first stabilisation member, and the second leg and a third leg are associated with the second stabilisation member.

It will be appreciated that any appropriate number of stabilisation members may be coupled together.

The stabilisation device may comprise a plurality of stabilisation members, wherein the stabilisation members are separable from one another.

### BRIEF DESCRIPTION OF THE FIGURES

Embodiments of the present invention will now be described, by way of example only, with reference to the accompanying figures, in which:

FIG. 1a is a posterior view of a portion of a spine having two orthopaedic stabilisation devices implanted therein in accordance with an embodiment of the present invention;

FIG. 1b is a sagittal view of the spine portion of FIG. 1a;

FIG. 2a is a perspective view of an orthopaedic stabilisation device in a contracted configuration in accordance with an embodiment of the present invention;

FIG. 2b is a perspective view of the orthopaedic stabilisation device of FIG. 2b in an expanded configuration;

FIG. 3a is side view of the orthopaedic stabilisation device of FIG. 2a;

FIG. 3b is a front view of the orthopaedic stabilisation device of FIG. 2a;

FIG. 3c is a top view of the orthopaedic stabilisation device of FIG. 2a;

FIG. 3d is a bottom view of the orthopaedic stabilisation device of FIG. 2a;

FIG. 3e is a cross sectional view of section A, as indicated in FIG. 3a, of the orthopaedic stabilisation device of FIG. 2a;

FIG. 3f is a cross sectional view of section B, as indicated in FIG. 3b, of the orthopaedic stabilisation device or FIG. 2a;

FIG. 4a is a perspective view of an orthopaedic stabilisation device in a contracted configuration in accordance with a further embodiment of the present invention;

FIG. 4b is a side view of the orthopaedic stabilisation device of FIG. 4a;

FIG. 4c is a top view of the orthopaedic stabilisation device of FIG. 4a;

FIG. 4d is a cross sectional view of section B, as indicated in FIG. 4b, of the orthopaedic stabilisation device of FIG. 4a;

FIG. 5a is a cross sectional view of a leg of an orthopaedic stabilisation device in a contracted configuration in accordance with an embodiment of the present invention;

FIG. 5b is a cross sectional view of the leg of FIG. 5a in an expanded configuration;

FIG. 6a is a cross sectional view of a leg of an orthopaedic stabilisation device in a contracted configuration in accordance with an embodiment of the present invention;

FIG. 6b is a cross sectional view of the leg of FIG. 6a in an expanded configuration;

FIG. 7a is a cross sectional view of a leg of an orthopaedic stabilisation device in accordance with an embodiment of the present invention;

FIG. 7b is a cross sectional view of a leg of an orthopaedic stabilisation device in accordance with an embodiment of the present invention;

FIGS. 8a to 8f are cross sectional views of actuating members of an orthopaedic stabilisation device in accordance with embodiments of the present invention;

FIG. 9 is a cross sectional view of an orthopaedic stabilisation device in accordance with an embodiment of the present invention;

FIG. 10 is a side view of a leg of an orthopaedic stabilisation device in accordance with an embodiment of the present invention;

FIG. 11 is a schematic diagram of various orthopaedic stabilisation devices in use stabilising adjacent vertebrae in accordance with an embodiment of the present invention;

FIGS. 12a to 12c are cross sectional views of a leg of an orthopaedic stabilisation device in accordance with an embodiment of the present invention;

FIG. 13a is a cross sectional view of a leg of an orthopaedic stabilisation device in accordance with an embodiment of the present invention;

FIG. 13b is a cross sectional view of various barb configurations for the leg of FIG. 13a;

FIG. 14 shows various cross sectional views of a leg of an orthopaedic stabilisation device in accordance with an embodiment of the present invention;

FIG. 15 is a cross sectional view of a leg of an orthopaedic stabilisation device in accordance with an embodiment of the present invention;

FIG. 16 is a cross sectional view of a leg of an orthopaedic stabilisation device in accordance with an embodiment of the present invention, the leg being shown to move between contracted and expanded configurations;

FIG. 17 is a cross sectional view of a leg of an orthopaedic stabilisation device in accordance with an embodiment of the present invention, the leg being shown moving from a contracted to an expanded configuration;

FIG. 18 shows various end views of legs of an orthopaedic stabilisation device in accordance with an embodiment of the present invention;

FIG. 19 shows partial perspective views of the legs of FIG. 18;

FIG. 20 is a cross sectional view of a leg of an orthopaedic stabilisation device in accordance with an embodiment of the present invention, the leg being shown in a contracted and an expanded configuration;

FIG. 21 shows cross sectional views of various legs of an orthopaedic stabilisation device in accordance with embodiments of the present invention, the legs being shown in contracted and expanded configurations;

FIG. 22 is a cross sectional view of a leg of an orthopaedic stabilisation device in accordance with an embodiment of the present invention, the leg being shown in a contracted and an expanded configuration;

FIG. 23a is a posterior view of a portion of a spine showing live springs of two orthopaedic stabilisation devices implanted therein in accordance with an embodiment of the present invention;

FIG. 23b is a sagittal view of the spine portion of FIG. 23a;

FIGS. 24a to 24c are top views of a stabilisation member of an orthopaedic stabilisation device in accordance with an embodiment of the present invention, the stabilisation member being shown in various stages of compression or expansion;

FIG. 25 is a partial cut away view of a stabilisation member of an orthopaedic stabilisation device in accordance with an embodiment of the present invention;

FIG. 26a is a perspective view of a stabilisation member of an orthopaedic stabilisation device in accordance with an embodiment of the present invention, the stabilisation member being arranged such that a stiffness mechanism is in series with a motion limit mechanism;

FIG. 26b shows top and bottom perspective views of a stabilisation member of an orthopaedic stabilisation device in accordance with an embodiment of the present invention, the stabilisation member being arranged such that a stiffness mechanism is in parallel with a motion limit mechanism;

FIG. 27 is a partial cross sectional view of a stabilisation member of an orthopaedic stabilisation device in accordance with an embodiment of the present invention;

FIG. 28 is a partial cross sectional view of a stabilisation member of an orthopaedic stabilisation device in accordance with an embodiment of the present invention;

FIG. 29 is a partial cross sectional view of a stabilisation member of an orthopaedic stabilisation device in accordance with an embodiment of the present invention;

FIG. 30 is a partial cross sectional view of a stabilisation member of an orthopaedic stabilisation device in accordance with an embodiment of the present invention;

FIG. 31 is a top view of a stabilisation member of an orthopaedic stabilisation device in accordance with an embodiment of the present invention;

FIG. 32 shows top and side partial cross sectional views of a stabilisation member of an orthopaedic stabilisation device in accordance with an embodiment of the present invention;

FIG. 33 shows various partial views of a stabilisation member of an orthopaedic stabilisation device in accordance with an embodiment of the present invention;

FIG. 34 is a top cross sectional view of a portion of a stabilisation member of an orthopaedic stabilisation device in accordance with an embodiment of the present invention;

FIGS. 35a and 35b show partial cross sectional views of stabilisation members of an orthopaedic stabilisation device in accordance with an embodiment of the present invention;

FIG. 36 shows top and side cross sectional views of a portion of an orthopaedic stabilisation device comprising a length varying mechanism;

FIG. 37 shows top and side cross sectional views of a portion of an orthopaedic stabilisation device comprising a length varying mechanism;

FIG. 38 shows a cross sectional view of a portion of an orthopaedic stabilisation device comprising a length varying mechanism;

FIG. 39 shows various views of single and multi-level stabilisation members of an orthopaedic stabilisation device in accordance with an embodiment of the present invention;

FIG. 40 is a partial side view of an orthopaedic stabilisation device that is arranged to be cut to size in accordance with an embodiment of the present invention;

FIG. 41 shows various views of stabilisation members of an orthopaedic stabilisation device in accordance with an embodiment of the present invention, the stabilisation members having a modular design to facilitate coupling the stabilisation members together; and

FIGS. 42a to 42e show various views of an awling tool in accordance with an embodiment of the present invention.

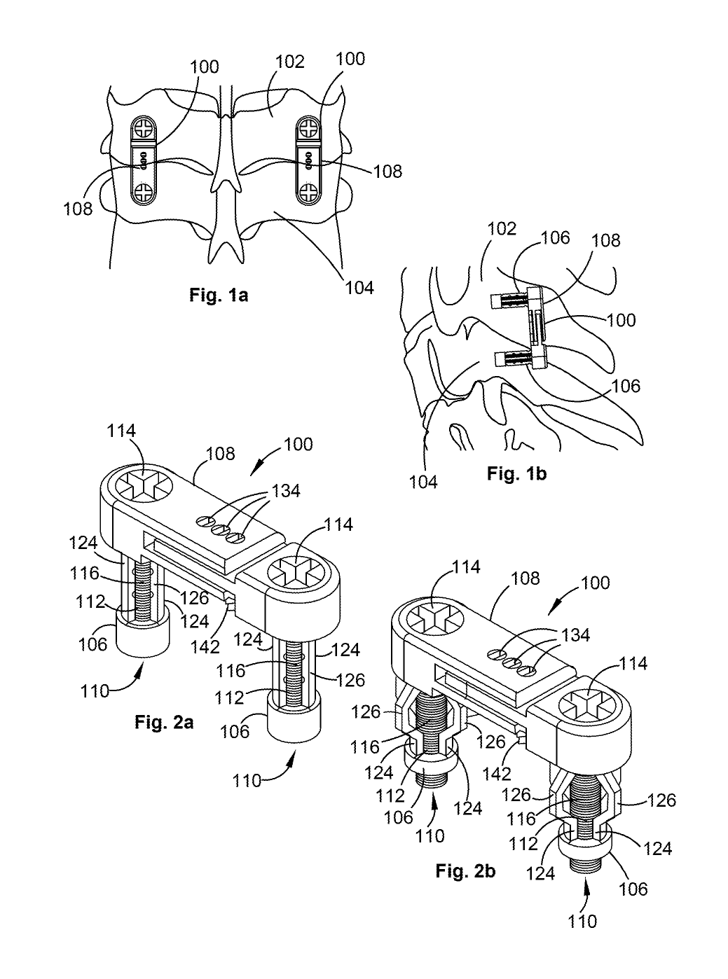

### DETAILED DESCRIPTION

In accordance with an embodiment of the present invention, there is provided an orthopaedic stabilisation device for use in facilitating the stabilisation of two or more bones, such as vertebrae, with respect to one another. The orthopaedic stabilisation device can also be used to stabilise two or more bone portions, such as fractured portions of a metatarsal bone.

The orthopaedic stabilisation device comprises at least two legs that are received in bore holes of respective bones, such as adjacent vertebrae, and a stabilisation member that bridges the two legs and facilitates stabilising the respective bones with respect to one another.

Each leg can be arranged to receive an element that results in fastening of the leg in its respective bore hole. The element that results in fastening of the leg in its respective bore hole may be a fastening element, such as a fastening screw, or may cause the leg to function as a fastening element, such as by causing the leg to expand and fasten in its respective bore hole.

The stiffness of the stabilisation member can be controlled, and a motion limit of the stabilisation member can be set, so as to provide dynamic stabilisation of the bones to which the orthopaedic stabilisation device is implanted. Such an orthopaedic stabilisation device can be used, for example, to stabilise two adjacent vertebrae whilst maintaining partial and controlled intervertebral motion.

The stabilisation member may also be static, which can prevent painful motion by restricting motion through fusion in conjunction with a graft.

The legs and the stabilisation member are arranged so as to facilitate insertion of the orthopaedic device into the bore holes in one piece. In this example, such an arrangement is achieved by integrating the legs and the stabilisation member. Such an arrangement obviates the need to align and adjust conventional orthopaedic fasteners and to couple the conventional orthopaedic fasteners to fusion instrumentation such as rods and plates that are used in conventional orthopaedic stabilisation devices.

The orthopaedic stabilisation device may be formed from titanium, or a material that promotes binding of the bone to the orthopaedic stabilisation device. The orthopaedic stabilisation device may also be formed from stainless steel, Delrin, polyetheretherketone or any other biocompatible material.

External surfaces of the orthopaedic stabilisation device may be relatively rough to facilitate the orthopaedic stabilisation device in engaging with the bone and to allow space for the bone to grow into the orthopaedic stabilisation device to facilitate effective osseointegration.

Example orthopaedic stabilisation devices 100 are shown in use stabilising first and second adjacent vertebrae 102, 104. In this example, the first and second adjacent vertebrae 102, 104 are cervical vertebrae corresponding to the C6 and C7 cervical vertebrae respectively.

The orthopaedic stabilisation devices 100 are implanted into respective lateral masses of the first and second vertebrae 102, 104, with one orthopaedic stabilisation device 100 on each lateral side.

Each orthopaedic stabilisation device 100 comprises two legs 106 and a stabilisation member 108. Each leg 106 is implanted into a respective vertebra 102, 104, and the stabilisation member 108 functions to stabilise the vertebrae 102, 104 with respect to one another.

In this embodiment, the legs 106 and the stabilisation member 108 are arranged to be insertable into the bore holes in one piece. This is achieved by integrating the legs 106 and the stabilisation member 108.

FIGS. 2a and 2b show an example embodiment of the stabilisation device 100 in more detail. Each leg 106 is moveable from a contracted configuration, as shown in FIG. 2a, to an expanded configuration, as shown in FIG. 2b. When in the contracted configuration, the legs 106 are receivable within a respective bore hole in bone. The legs 106 are moveable to the expanded configuration when located in the bore hole to facilitate fastening each leg 106 within its respective bore hole.

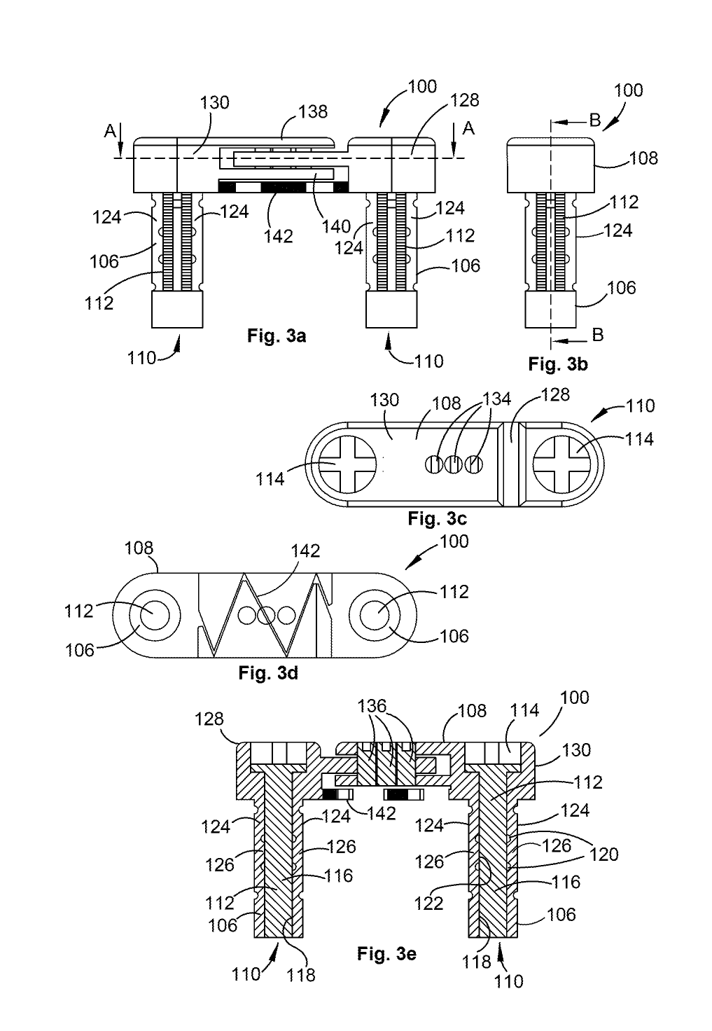

Referring also to FIGS. 3a to 3f, the legs 106 each comprise a passage 110 that is arranged to receive an actuating member 112. The actuating members 112 are, in this example, threaded screws that are arranged, when rotated in a first direction, to urge the legs 106 towards the stabilisation member 108, thereby causing the legs 106 to move from the contracted configuration of FIG. 2a to the expanded configuration of FIG. 2b.

To facilitate this action, each actuating member 112 comprises a head portion 114 arranged to receive a hex-head screw driver or other tools for imparting rotation to the actuating member 112, and a threaded body portion 116. An internal surface 118 of a lower end of each leg 106 is threaded so as to engage with the threaded body portion 116.

As each leg 106 is urged towards the stabilisation member 108, the leg 106 will buckle at predefined locations. The buckling is facilitated by notches 120 arranged at predefined locations along an internal surface 122 of each of a plurality of leg struts 124 of each leg 106.

In this example, rotating the actuating member 112 in an opposite direction to the first direction will urge each leg 106 to move from the expanded configuration of FIG. 2b to the contracted configuration of 2a. This can facilitate removal of the orthopaedic stabilisation device 100 if desired, and provides an orthopaedic stabilisation device 100 that is moveable between the contracted and expanded configurations.

Moving each leg 106 to the expanded configuration increases a radial dimension of the leg 106 compared to when the leg 106 is in the contracted configuration, and facilitates retaining the leg within its respective bore hole. In this example, when the leg 106 moves to the expanded configuration, a middle portion 126 of each leg strut 124 is arranged to have an external surface that is substantially parallel to an axis of each leg 106, thereby increasing a surface area of each leg 106 that is in contact with bone. Such an arrangement can increase a pull-out strength of the orthopaedic stabilisation device 100.

The stabilisation member 108 is arranged such that a length of the stabilisation member 108 is alterable. In this example, the stabilisation member 108 comprises a first stabilisation portion 128 and a second stabilisation portion 130, wherein the first and second stabilisation portions 128, 130 can move relative to one another.

An amount by which the stabilisation portions 128, 130 can move relative to one another is constrained by a predefined amount, and hence an amount by which the length of the stabilisation member 108 can be altered is constrained. Constraining the relative motion of the first and second stabilisation portions 128, 130 is achieved in this example by providing an elongate slot 132 (see FIG. 3f) in the first stabilisation member that can engage with one or more pins 134 that can be inserted through, or removed from, respective apertures 136 provided in the second stabilisation portion 130.

Inserting a pin 134 into, or removing a pin from, different apertures 136 will provide different ranges of motion. It will also be appreciated that the first and second stabilisation portions 128, 130 can be prevented from moving relative to one another by inserting a pin 134 into each aperture 136, or at least into the apertures 136 that correspond with ends of the elongate slot 132.

In this example, the second stabilisation portion 130 comprises an upper plate 138 and a lower plate 140, the elongate slot 132 of the first stabilisation portion 128 being received therebetween. The upper and lower plates 138, 140 each comprise the apertures 136 for receiving the pins 134, thereby increasing a stability of the orthopaedic stabilisation device 100. However, to simplify manufacturing the device may be constructed using only one plate with an aperture and one slotted plate.

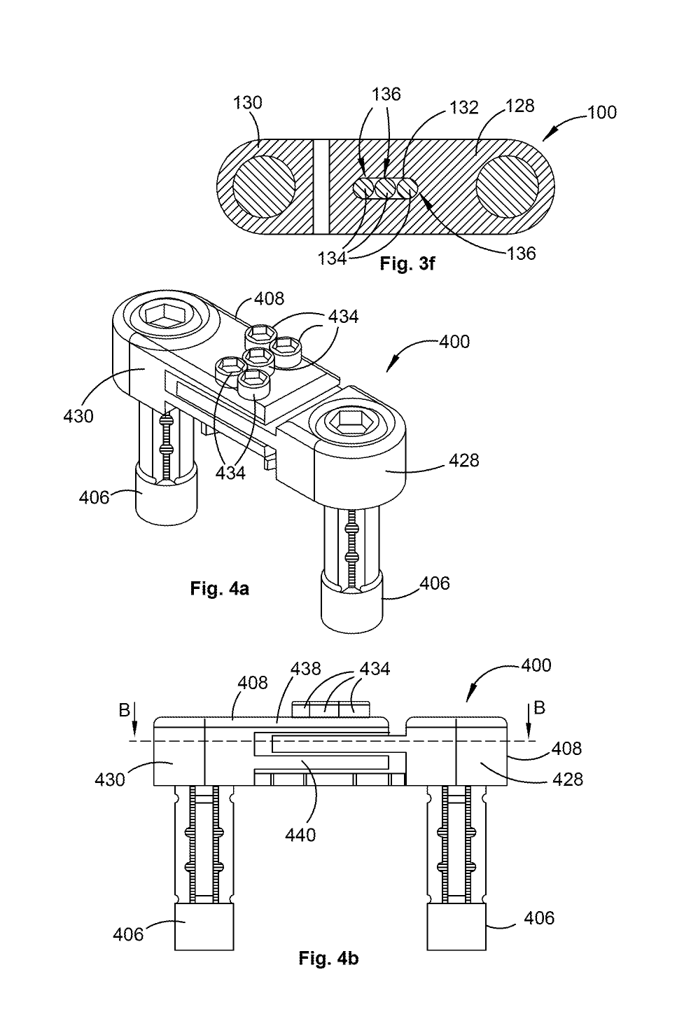

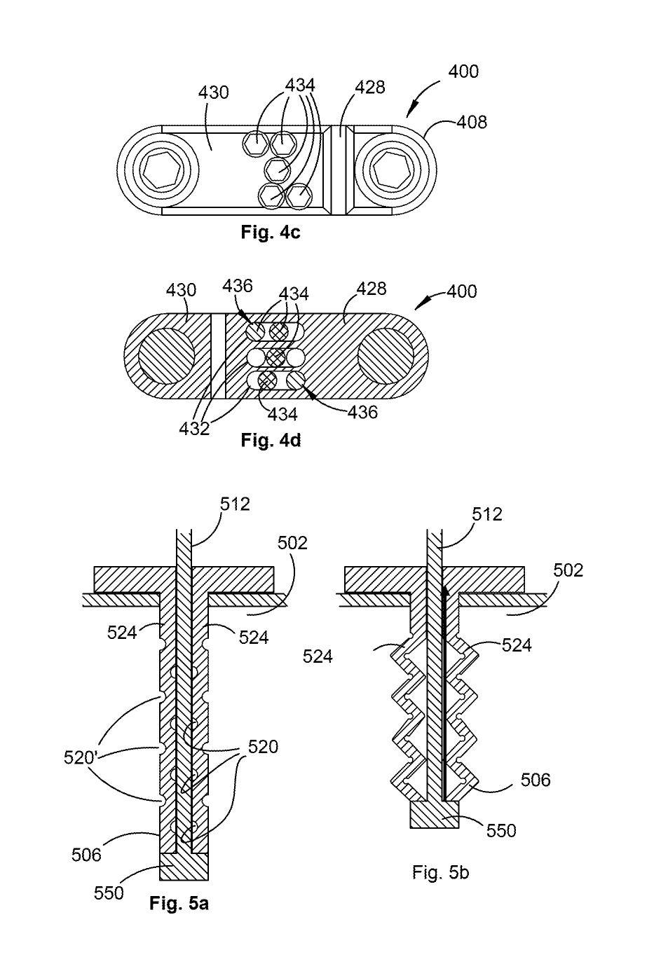

It will be appreciated that other slot and pin configurations can be used to provide different motion constraint options to the orthopaedic stabilisation device. For example, and as shown in FIGS. 4a to 4d, there is shown an orthopaedic stabilisation device 400 having an alternative slot and pin configuration to that of orthopaedic stabilisation device 100. The orthopaedic device 400 is similar to the orthopaedic device 100, and comprises two legs 406 and a stabilisation member 408. The stabilisation member 408 comprises a first stabilisation portion 428 and a second stabilisation portion 430, the first and second stabilisation portions 428, 430 being moveable relative to one another.

In this example, the first stabilisation portion 428 comprises three elongate slots 432 (see FIG. 4d) that are arranged to receive pins 434, and the second stabilisation portion 430 comprises a plurality of apertures 436 that are arranged to receive or reject the pins 434. With this arrangement, pins 434 can be placed as desired to constrain both linear and rotational motion of the first and second stabilisation portions 428, 430 with respect to one another. As with the orthopaedic stabilisation device 100, the second stabilisation portion 430 comprises upper and lower plates 438, 440 so as to increase a stability of the orthopaedic stabilisation device 400. However, to simplify manufacturing the device may be constructed using only one plate with an aperture and one slotted plate. Furthermore any number of elongate slots 432 can be used to strike a balance between simplifying manufacturing and providing more choice in range of motion.

Referring back to FIGS. 3a to 3f, the orthopaedic stabilisation device 100 also comprises a live spring 142 that couples the first and second stabilisation portions 128, 130 to one another in addition to the coupling between the elongate slot 132 and the pin(s) 134. The live spring 142 facilitates control of a stiffness of the stabilisation member 108, and can assist in reducing impulse loading of the legs 106 and preventing confounding relative motion of the legs 106 during implantation. Further, when the orthopaedic stabilisation device 100 is used to stabilise vertebrae, the live spring 142 can assist in reducing motion and loading of facet joints of the vertebrae during small movements of the spine to minimise incidence or severity of facet arthritis.

If a stiffness control mechanism, such as the live spring 142, is not provided, then impulse loading of the legs 106 caused by free movement of the stabilisation member 108 may lead to accelerated loosening of the legs 106 from their respective bore holes. Further, if the orthopaedic stabilisation device 100 can freely move throughout its predefined variable length during implantation, then aligning the legs 106 to their respective bore holes may present a challenge to a surgeon performing the implantation. Finally, in the case of facet arthritis, minimising movement and loading sharing at the facet joints during micro-motions of the vertebrae can facilitate treating, and preventing further, degeneration of the facet joints. However if these problems can be solved without using a spring then the orthopaedic stabilisation device 100 can be constructed without a spring also.

In this example, the live spring 142 is formed from an appropriate metal or metal alloy and the live spring 142 is bent in a zig-zag fashion in a plane that is parallel to section A-A.

The orthopaedic stabilisation devices 100, 400 represent just two example embodiments, and features of the orthopaedic stabilisation devices 100, 400 can be implemented in many different ways. Further example features of orthopaedic devices will now be described.

The legs 106, 406 of orthopaedic devices 100, 400 can be arranged to move from the contracted configuration to the expanded configuration, or between the contracted and expanded configurations, in many different ways to facilitate fastening the legs 106, 406 in their respective bore holes.

Referring to FIG. 5a, there is shown an example leg 506 that is arranged to move from a contracted configuration (FIG. 5a) to an expanded configuration (FIG. 5b) in a buckling action in response to movement of an actuating member 512 in a direction out of bone 502 that the leg 506 is inserted into.

The leg 506 comprises a plurality of notches 520 arranged on an internal surface of each leg strut 524, and a plurality of notches 520′ arranged on an external surface of each leg strut 524. The notches 520, 520′ facilitate each leg strut 524 buckling in a predetermined manner when the actuating member 512 moves out of the bone 502. In this example, the actuating member 512 comprises an end portion 550 that is arranged to engage with a remote end of the leg 506 and to urge the leg 506 into the expanded configuration shown in FIG. 5b.

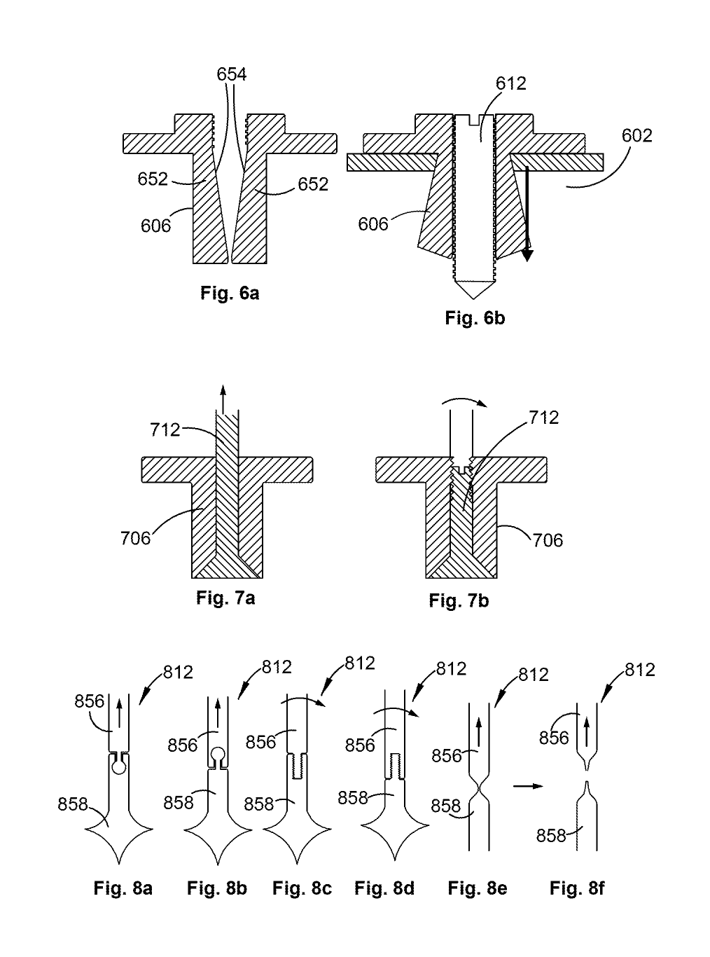

Referring to FIG. 6a, there is shown an example leg 606 that is arranged to move from a contracted configuration (FIG. 6a) to an expanded configuration (FIG. 6b) in a cantilever action in response to movement of an actuating member 612 in a direction into bone 602 that the leg 606 is inserted into.

The leg 606 comprises leg portions 652 having respective angled internal surfaces 654 that are arranged to be urged outwards when the actuating member 612 is moved in a direction into the bone 602, thereby moving the leg 606 into the expanded configuration as shown in FIG. 6b.

FIGS. 7a and 7b illustrate different methods of urging legs 706 from the contracted to the expanded configuration. In FIG. 7a, an actuating member 712 can be moved linearly, such as by an upwards pulling action or a downwards pressing action, to urge the leg 706 into the expanded configuration. In FIG. 7b, an actuating member 712 can be rotated to urge the leg 706 into the expanded configuration.

For embodiments wherein the actuating member 712 is moved linearly upwards to effect expansion of the leg 706, a portion of the actuating member 712 may be arranged to be removable. This can prevent the actuating member 712 from protruding from the orthopaedic stabilisation device 100. Example embodiments of such an arrangement are shown in FIGS. 8a to 8f.

An upper portion 856 of an actuating member 812 can be removed from a lower portion 858 of the actuating member 812 by a snap fit disconnection (FIGS. 8a and 8b), a threaded disconnection (FIGS. 8c and 8d) or a permanent rupture (FIGS. 8e and 8f).

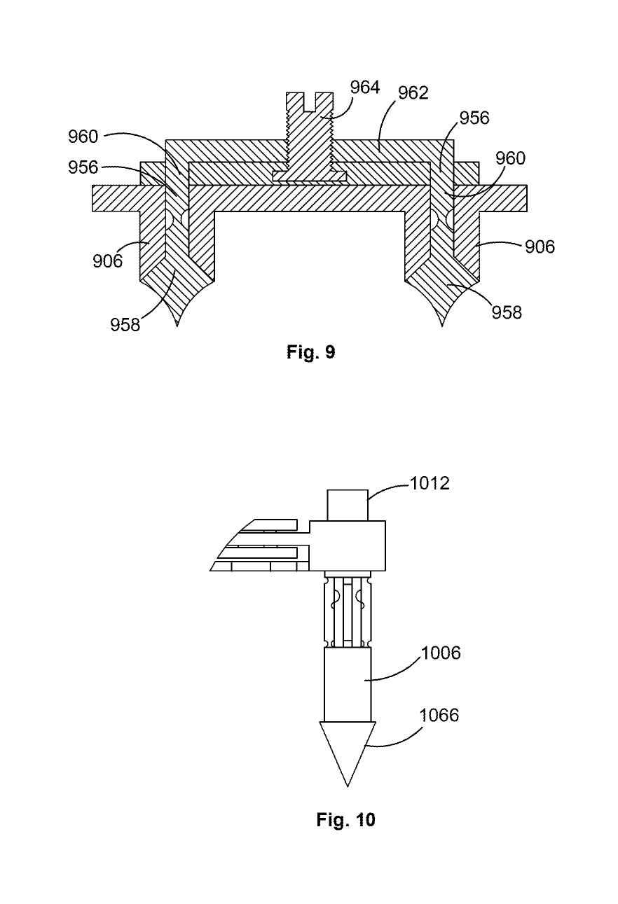

It will be appreciated that the legs 106 of the orthopaedic stabilisation device 100 can be moved from the contracted to the expanded configuration simultaneously or separately. Separate expansion of each leg 106 can be achieved by separately moving respective actuating members 112, for example with a screw driver having an appropriate head profile or a specially designed tool. An example of an arrangement whereby simultaneous expansion of legs 906 can be effected is illustrated in FIG. 9.

In this example, an actuating member 912 is provided that comprises two leg portions 960 that are arranged to be received by respective legs 906, and that are coupled together by a bridge portion 962. Both leg portions 960 can be moved upwards in one action to expand the legs 906 simultaneously by pulling a handle portion 964 upwards either directly or through a threaded advancement caused by revolving a threaded member. In this example, each leg portion 960 comprises a lower portion 958 and an upper portion 956, the upper portion 956 being removable from the lower portion 958 in a similar manner to that as shown in FIGS. 8e and 8f.

Providing an arrangement whereby the legs 906 can be expanded simultaneously can assist in reducing surgical time when implanting the orthopaedic stabilisation device 100 and may reduce difficulty in lining up the orthopaedic stabilisation device 100 prior to expanding the legs 906.

FIG. 10 shows an example leg 1006 that is arranged to receive an actuating member 1012 having an awling tip 1066. The awling tip 1066 can assist in implanting the orthopaedic stabilisation device 100, as the awling tip 1066 can be used to punch the bore hole in the bone to which the leg 1006 is to be fastened. It will be appreciated, however, that any suitable device can be used to create the bore hole, such as a separate awling tool or drill.

It will be appreciated that, although the above examples relate to an orthopaedic stabilisation device 100 having two legs 106, any number of legs 106 can be provided. For example, FIG. 11 shows a variety of top views of orthopaedic stabilisation devices 100 being used to stabilise first and second vertebrae 102, 104. The examples shown in FIG. 11 illustrate orthopaedic stabilisation devices 100 having two, four, and six legs 106.

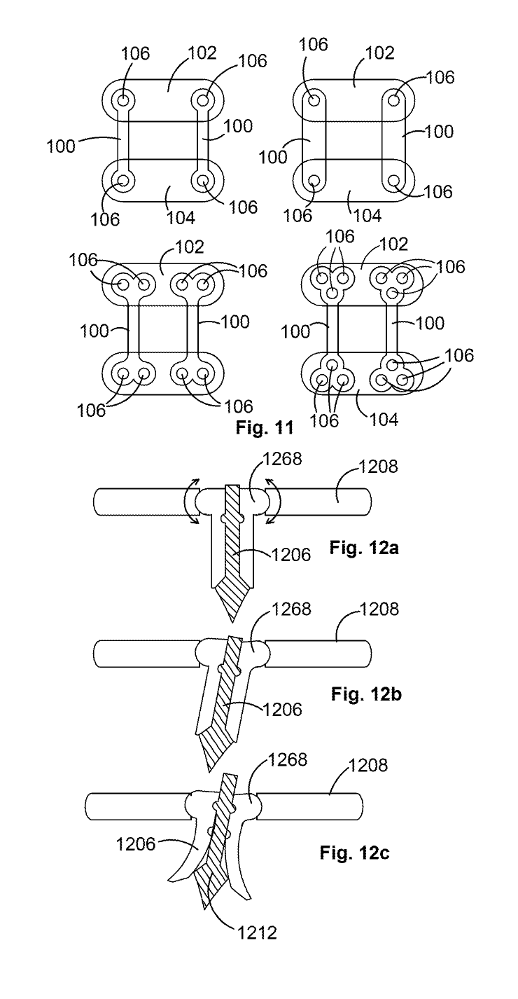

In some embodiments, an angle of each leg 106 with respect to a plane of the stabilisation member 108 can be arranged to be varied as desired. For example, and as shown in FIGS. 12a to 12c, an angle of a leg 1206 can be varied using a locking plate mechanism. The locking plate mechanism works by expanding a rotational portion 1268 of the leg 1206 to press fit into a plate to which the leg 1206 is coupled, such as a portion of the stabilisation member 1208. In this example, the rotational portion 1268 is expanded when an actuating member 1212 is moved upwards, for example when expanding the leg 1206. Expansion of the rotational portion 1268 causes friction or interference between the rotational portion 1268 and the stabilisation member 1208 to which the rotational portion 1268 is coupled, locking the orientation of the leg 1206.

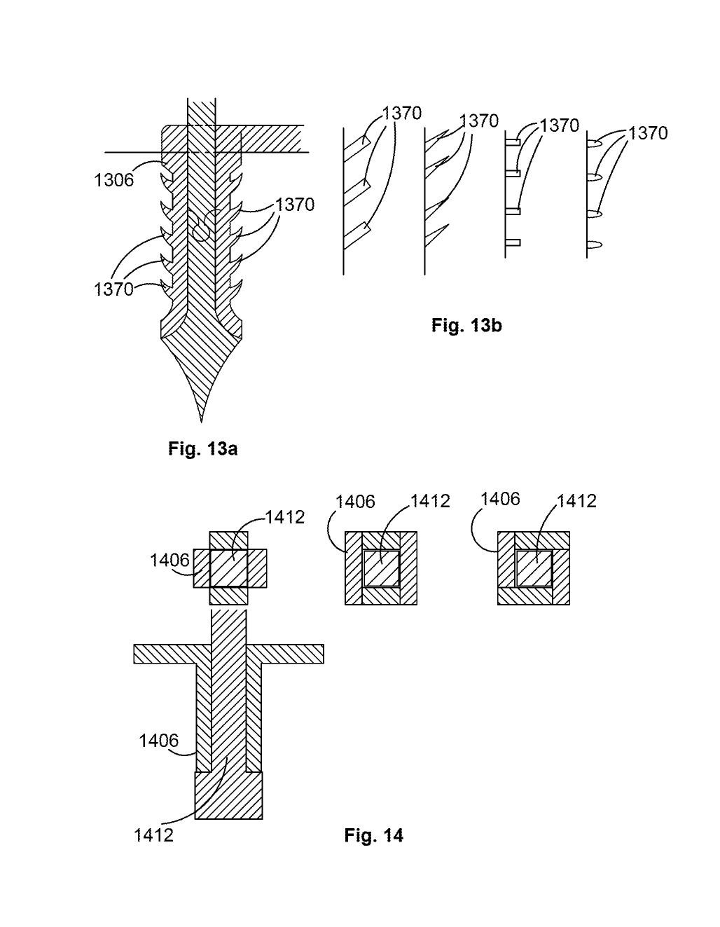

Each leg 106 may comprise a plurality of barbs. An example leg 1306 comprising a plurality of barbs 1370 is shown in FIG. 13a. The barbs 1370 can increase friction between the leg 1306 and bone into which the leg 1306 is implanted, and are elastically retracted during implantation. The barbs 1370 may be any appropriate shape, examples of which are shown in FIG. 13b. Blunt barbs 1370, such as barbs 1370 having an elliptical profile, may assist in preventing stress concentration, crack initiation and eventual fatigue failure.

The axial profile of each leg 106 and their respective actuating members 112 can be any appropriate shape, such as circular, triangular or square. Some axial profiles, such as a circular profile, may provide manufacturing benefits. Non-rotationally symmetric profiles, such as a square profile, may provide benefits when implanted in bone as they can facilitate preventing rotation. Example legs 1406 and actuating members 1412 having square profiles are shown in FIG. 14.

The action by which the legs 106 expand can be any appropriate action. The example orthopaedic stabilisation devices 100, 400 described earlier are arranged to expand by a buckling action in response to the actuating member 112 moving in a direction out of the bone. In the examples, the legs 106 comprise four leg struts 124 that have notches 120 to facilitate buckling of the leg struts 124 at the locations of the notches 120. It will be appreciated that any number of leg struts 124 and/or notches 120 can be provided. Providing a plurality of notches 120 on a leg strut 124 can provide a leg 106 that has multiple stages of expansion.

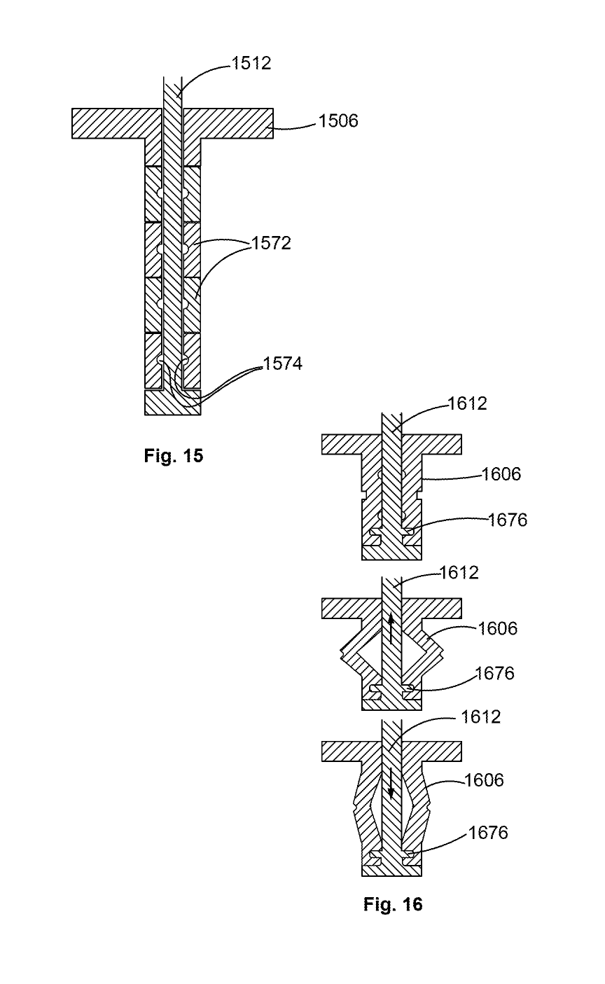

It will be appreciated that other arrangements for achieving expansion of the legs 106 are envisaged. An alternative arrangement is illustrated in FIG. 15. In this example, a leg 1506 comprises a plurality of washers 1572, each washer having a notch 1574 to facilitate buckling of the washer 1572. The washers 1572 are separable, and can be coupled together by respectively threaded end portions, thereby providing a leg 1506 that has a length that can be set as desired.

As described earlier, the legs 106 can be arranged so as to be moveable between the contracted and expanded configurations. An example of a mechanism for facilitating this type of arrangement is shown in FIG. 16. In this example, a lower portion 1676 of an actuating member 1612 is arranged to be received by a correspondingly shaped region of a leg 1606 such that the actuating member 1612 can be moved upwards and downwards to effect moving the leg 1606 between the contracted and expanded configurations.

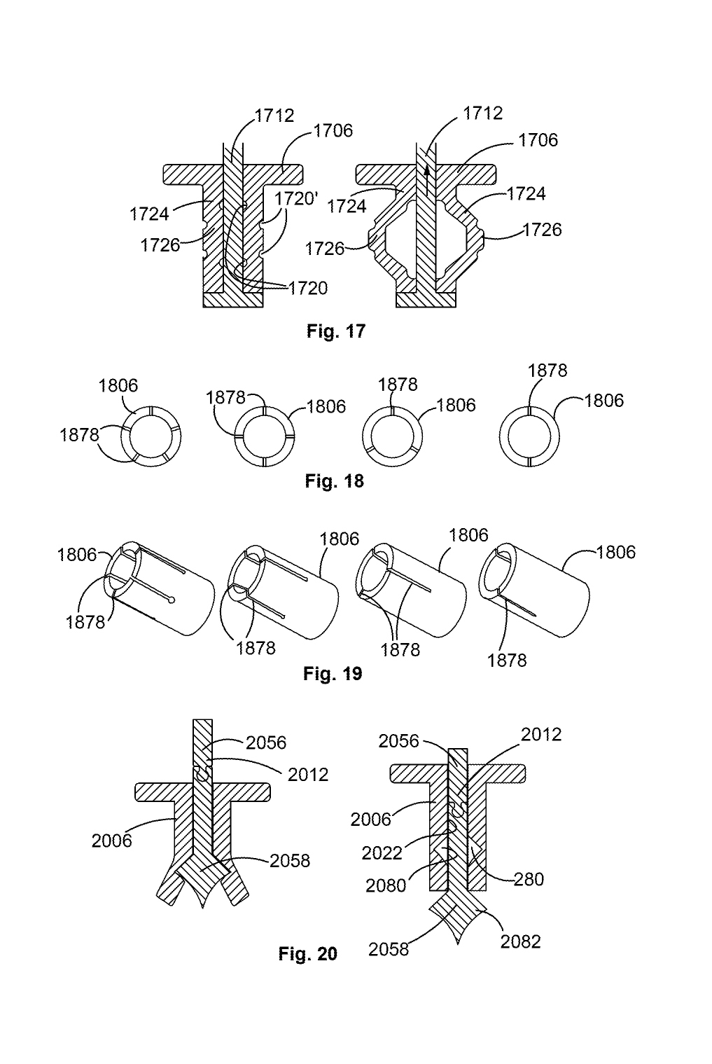

Further, and as described earlier, each leg 106 may be arranged such that, when expanded, a middle portion 126 of each leg strut 124 is arranged to have an external surface that is substantially parallel to an axis of each leg 106, thereby increasing a surface area of each leg 106 that is in contact with bone. An example of such an arrangement is illustrated in FIG. 17. In this example, internal notches 1720 and external notches 1720′ of each leg strut 1724 are arranged such that a middle portion 1726 of each leg strut is substantially parallel to an axis of each leg 1706 when the actuating member 1712 is moved upwards and the leg 1706 moves to the expanded configuration.

As described earlier with reference to FIG. 6, the legs 106 may be arranged to expand in a cantilever action. FIGS. 18 and 19 illustrate example configurations of portions of a leg 1806 that can facilitate cantilever deformation as the leg 1806 moves to the expanded configuration. The legs 1806 may comprise any number of longitudinal slots 1878. Increasing the number of longitudinal slots 1878 will reduce the force needed to expand the legs 1806.

With arrangements wherein the legs 106 are arranged to expand in a cantilever action, an internal surface 2022 of a leg 2006 can be provided with snap fit grooves 2080 that have a complementary shape to an outer portion 2082 of a head of the actuating member 2012. In this way, the outer portion 2082 can be retained in the grooves 2080 when the actuating member 2012 is moved upwards and the leg 2006 is moved to the expanded configuration, thereby preventing the actuating member 2012 from backing out of the leg 2006 and moving the leg 2006 back to the contracted configuration, or a partially contracted configuration. Furthermore the snapping sound of the internal surfaces impacting the snap fit grooves will give confidence to the surgeon that the fastener has been sufficiently fastened, preventing over and under tightening of the device. In this example, the actuating member 2012 comprises separable upper and lower portions 2056, 2058.

The legs 106 may be arranged to expand when the actuating member 112 is inserted into the passage 110 to provide a friction fit into bone. For example, and as shown in FIG. 21, a leg 2106 may comprise a passage 2110 that decreases in diameter along a length of the leg 2106, or a passage that has a constant diameter along the length of the leg 2106 that is smaller than that of the actuating member 2112. An actuating member 2112 can be inserted into the passage 2110, thereby causing the leg 2106 to expand so as to form an interference fit with the surrounding bone. An upper portion 2184 of the actuating member 2112 may protrude outwards and can be retained in a correspondingly shaped groove 2186 of the leg 2106 when the actuating member 2112 has been inserted into the leg 2106 and the leg 2106 has moved to the expanded configuration, thereby preventing the actuating member 2112 from backing out of the leg 2106 and moving back to the contracted configuration, or a partially contracted configuration.

A threaded actuating member 2112′ can be provided, and a passage 2110′ of a leg 2106′ can be arranged to expand when the actuating member 2112′ is inserted into the passage 2110′. An internal surface of the passage 2110′ can be threaded to facilitate insertion of the actuating member 2112′. Alternatively, the internal surface of the passage 2110′ can be unthreaded and formed from a softer material than the actuating member 2112′, wherein the actuating member 2112′ threads into the internal surface of the passage 2110′ when inserted into the passage 2110′. Alternatively the actuating member 2112′ can be non-threaded and the expansion action can comprise a linear impact, such as from a hammer or a linear pull such as for a pot-rivet.

In both cases, an external surface of the legs 2106, 2106′ can be roughened, and/or may be provided with barbs, ridges, or spikes to facilitate the interference fit with the surrounding bone.

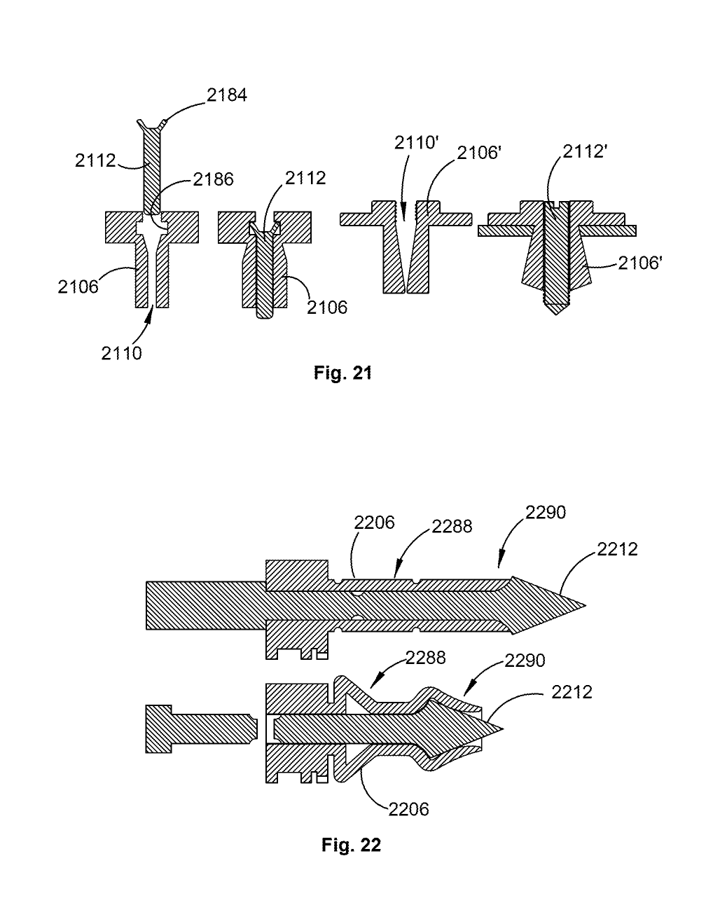

The legs 106 may be arranged to use a combination of various expanding actions. For example, and as shown in FIG. 22, a leg 2206 may comprise a first portion 2288 that is arranged to move to the expanded configuration in a buckling action in response to movement of the actuating member 2212, similar to the buckling action described with reference to FIG. 5, and a second portion 2290 that is arranged to move to the expanded configuration in a cantilever action, similar to the cantilever action described with reference to FIG. 6. The first portion 2288 can provide effective clamping of the leg 2206 to a cortical bone of a vertebra, and the second portion 2290 can prevent wastage of a lower region of the leg 2206 and increase a fixation with cancellous bone of a vertebra.

Referring back to FIGS. 2a, 2b and 3a to 3f, the stabilisation member 108 will now be described in more detail.

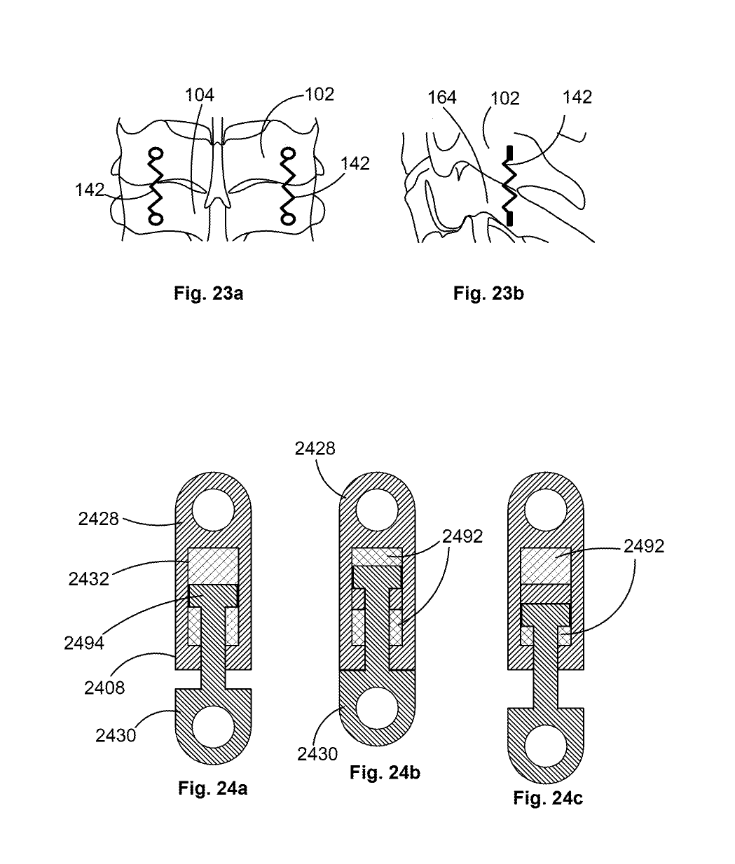

The live spring 142 of the stabilisation member 108 can be bent in the coronal plane and/or the sagittal plane, as shown in FIGS. 23a and 23b. The live spring 142 can also have no bends, or it may have one or more bends. The profile of the bends may be any appropriate shape, such as square, circular or triangular. A continuous profile, such as a circular profile, may reduce stress concentration, increasing fatigue life. In contrast, square bends are inherently less stiff and can provide more extension and compression for a given active length.

A cross-sectional profile of the live spring 142 may be any appropriate shape, such as elliptical, rectangular, square, circular or triangular.

Although a single live spring 142 is provided in this example, it will be appreciated that any number of springs or flexible members can be used. An increased number of springs can increase a stability and stiffness of the stabilisation member 108.

Other mechanisms for controlling a stiffness of the stabilisation member 108 are envisaged. For example, and with reference to FIGS. 24a to 24c, the stiffness of a stabilisation member 2408 can be controlled by compressible elements 2492 that are arranged in a slot 2432 of a first stabilisation portion 2428 of the stabilisation member 2408. A plate member 2494 of a second stabilisation portion 2430 will interact with, and meet resistance from, the compressible elements 2492 as the second stabilisation portion 2430 moves towards the first stabilisation portion 2428 (FIG. 24b) or away from the first stabilisation portion 2428 (FIG. 24c) from a neutral position (FIG. 24a).

The compressible elements 2492 can be made from any suitable material, such as rubber, polymers, or any other elastic material. There may be any number of compressible elements 2492, and the compressible elements 2492 can be used in series or in parallel. The compressible elements 2492 can be integrated together to increase stability and/or to simplify manufacture of the compressible elements 2492. The compressible elements 2492 can be formed in any appropriate shape so as to modify the force required to deform the compressible elements 2492.

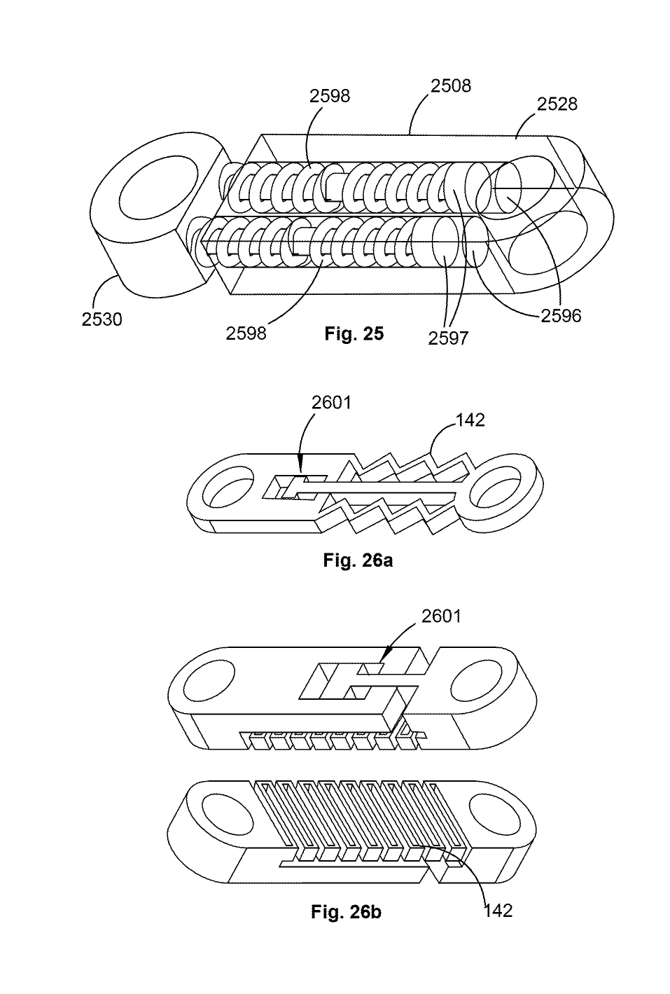

A stiffness of the stabilisation member 108 can also be controlled by using curved helical springs, as illustrated in FIG. 25. In this example, a first stabilisation portion 2528 of a stabilisation member 2508 comprises two passages 2596 for receiving respective legs 2597 of a second stabilisation portion 2530 of the stabilisation member 2508. A helical spring 2598 is coiled around each leg 2597 to provide stiffness control to the stabilisation member 2508. The helical springs 2598 can be any appropriate cross-sectional shape, such as elliptical, rectangular, square, circular or triangular. The helical springs 2598 can have any appropriate number of coils, wire thickness, coil diameter and can be formed from any appropriate material.

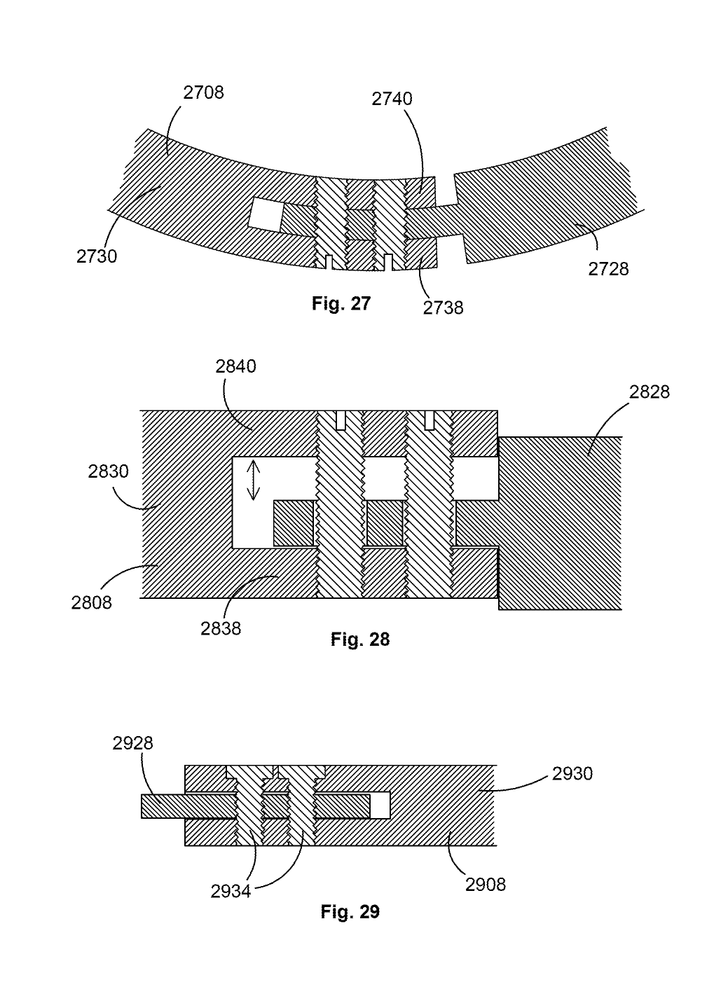

As discussed earlier, the stabilisation member 108 also functions to define limits of motion of the orthopaedic stabilisation device 100. The stiffness control mechanism, such as that provided by the live spring 142, can be in series (see FIG. 26a) or in parallel (see FIG. 26b) with the mechanism that is used to define the limits of motion of the orthopaedic stabilisation device 100. In the examples of FIGS. 26a and 26b, a motion limit control mechanism 2601 is provided by a ‘plate-in-plate’ configuration as described later with reference to FIG. 31.

It will be appreciated that the motion limit control function could be provided by the stiffness control mechanism, such as by the live spring 142 and so a separate mechanism to define the limits of motion is not essential.

The stabilisation member 108 may have a profile that more accurately imitates spinal motion compared to planar first and second stabilisation portions 128, 130. An example of such an arrangement is shown in FIG. 27, wherein first and second stabilisation portions 2728, 2730 of a stabilisation member 2708 are arcuate. Upper and lower plates 2738, 2740 of the second stabilisation portion 2730 are also arcuate.

Alternatively, if a truly physiological path of motion is not essential the stabilisation member 108 can also be arranged to facilitate at least some rotation of the first and second stabilisation portions 128, 130 relative to one another to approximate physiological movement. In one example, shown in FIG. 28, upper and lower plates 2838, 2840 of a second stabilisation portion 2830 or a stabilisation member 2808 are arranged such that there is clearance when a first stabilisation portion 2828 is received therebetween, thereby facilitating rotation of the first and second stabilisation portions 2828, 2830 with respect to one another.

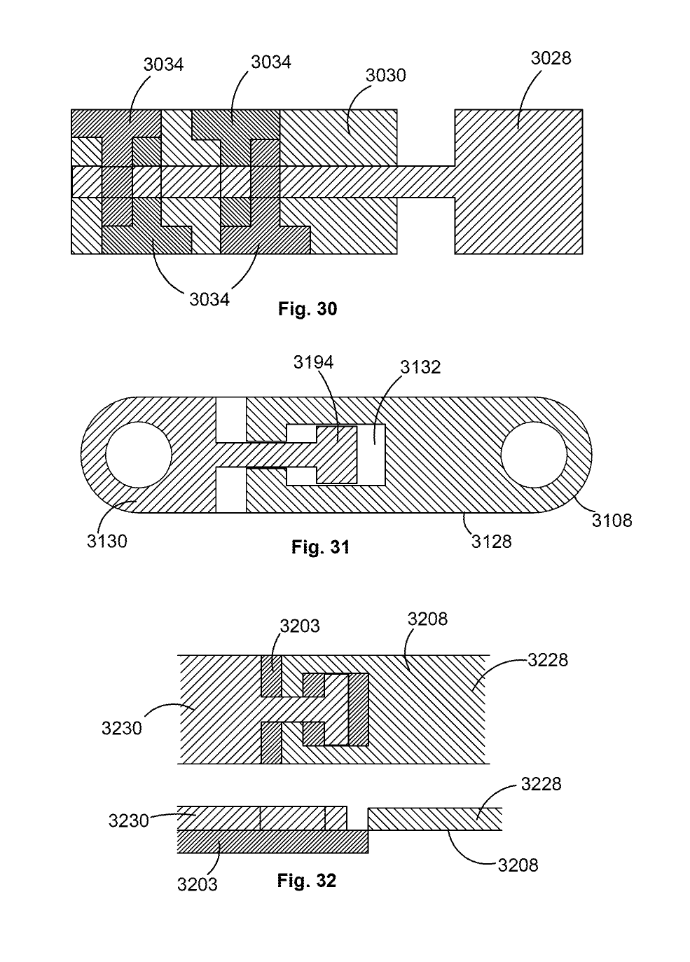

In the example orthopaedic stabilisation devices 100, 400, the motion limits are defined by a slot and pin mechanism. As shown in FIG. 29, pins 2934 used to couple together first and second stabilisation portions 2928, 2930 of a stabilisation member 2908 may have a larger diameter at an end that is adjacent an upper end of the stabilisation member 2908 so as to facilitate easier turning of the pins without increasing a central diameter of the pins 2934, thereby reducing space used and allowing a finer adjustment of range of motion of the orthopaedic stabilisation device 100. Further, multiple pins can be used to allow for varying degrees of flexion and extension and, as shown in FIG. 30, adjacent pins 3034 can be arranged in a ‘head to toe’ configuration so as to prevent interference between adjacent heads of pins 3034.

It will be appreciated that other mechanisms can be used to define the motion limits of the orthopaedic stabilisation devices 100, 400. For example, and as shown in FIG. 31, a ‘plate-in-plate’ arrangement can be used wherein an elongate slot 3132 of a first stabilisation portion 3128 is arranged to receive a plate member 3194 of a second stabilisation portion 3130. The plate member 3194 is constrained to movement within the elongate slot 3132. The first and second stabilisation portions 3128, 3130 can be in the same plane so as to allow for free rotation, or first and second stabilisation portions 3228, 3230 can be mounted with respect to a backing plate 3203 (see FIG. 32) so as to prevent non-axial motion. Clearance between the backing plate 3203 and the first and second stabilisation portions 3228, 3230 can be predefined so as to define an allowable degree of rotation to approximate spinal motion.

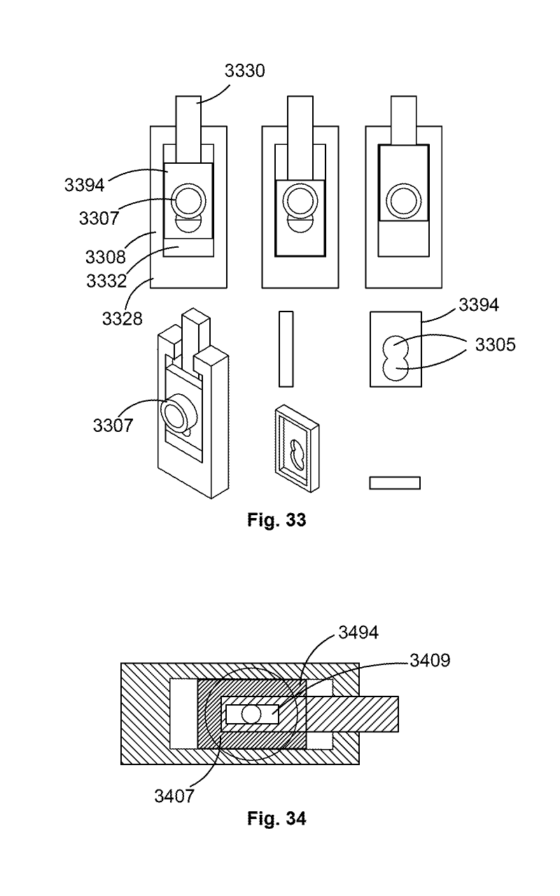

Referring to a stabilisation member 3308 shown in FIG. 33, a position of a plate member 3394 relative to a second stabilisation member 3330 can be adjustable. The plate member 3394 is received in an elongate slot 3332 of a first stabilisation member 3328, and adjusting the position of the plate member 3394 relative to the second stabilisation member 3330 can allow the stabilisation member 3308 to cater for varying ranges of motion. In this example, the plate member 3394 comprises two apertures 3305 that can receive a clamp member 3307 for clamping the plate member 3394 into a desired position relative to the second stabilisation member 3330. This can allow the stabilisation member 3308 to be arranged to allow movement corresponding to flexion and/or extension.

Whereas the example of FIG. 33 shows the plate member 3394 having discretely adjustable positions, FIG. 34 shows an example embodiment wherein a plate member 3494 can be clamped by clamp member 3407 at any desired position along an elongate slot 3409 of the plate member 3494.

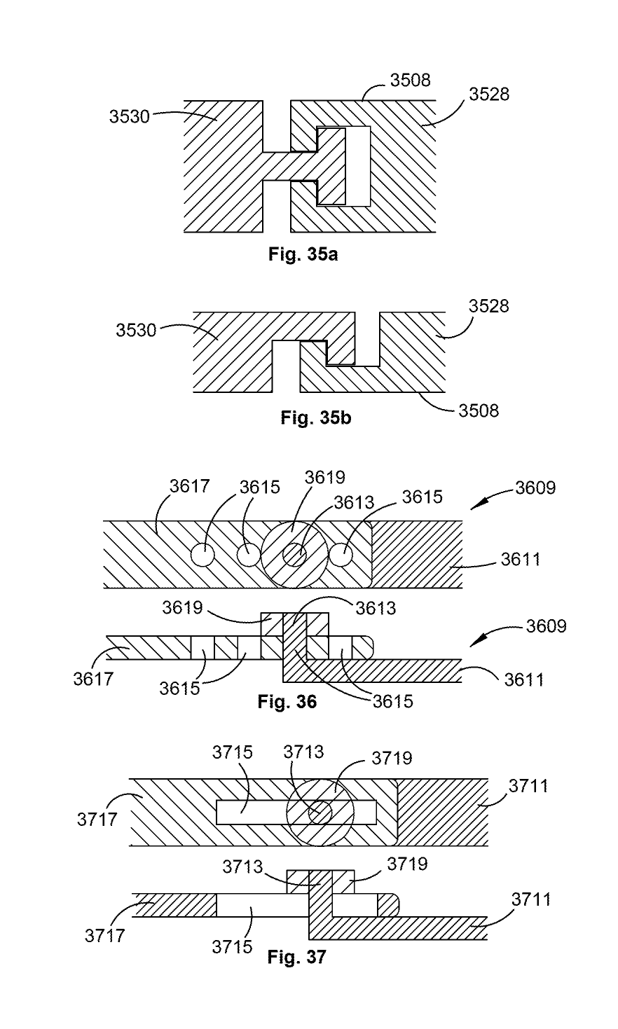

It will be appreciated that the ‘plate-in-plate’ arrangement may be configured such that multiple interference points between first and second stabilisation portions 3528, 3530 of a stabilisation member 3508 are be provided (see FIG. 35a), or wherein a single interference point between the first and second stabilisation portions 3528, 3530 is provided (see FIG. 35b), depending on the requirements for stability of the stabilisation member 3508.

It will be appreciated that the ideal distance between the legs 106 of the orthopaedic stabilisation device 100 may vary depending on the anatomy of the patient. As such, a distance between the legs 106 can be varied, such as by providing an adjustment mechanism or similar, or different orthopaedic stabilisation devices 100 can be provided having different spacing between legs 106.

As shown in FIG. 36, any appropriate portion 3609 of an orthopaedic stabilisation device can comprise a length varying mechanism that is in series with the motion limit control mechanism and the stiffness control mechanism. In this example, the portion 3609 of the orthopaedic stabilisation device comprises a first spacing portion 3611 comprising a protrusion 3613. The protrusion 3613 is receivable in one of a plurality of apertures 3615 of a second spacing portion 3617, and can be retained by nut 3619. In this example, a length of the orthopaedic stabilisation device, and therefore a spacing between the legs 106, is discretely alterable.

An alternative wherein a continuous adjustment of orthopaedic stabilisation device length is provided is shown in FIG. 37. In this example, a protrusion 3713 of a first spacing portion 3711 is receivable in an elongate slot 3715 of a second spacing portion 3717, and is retained by a nut 3719.

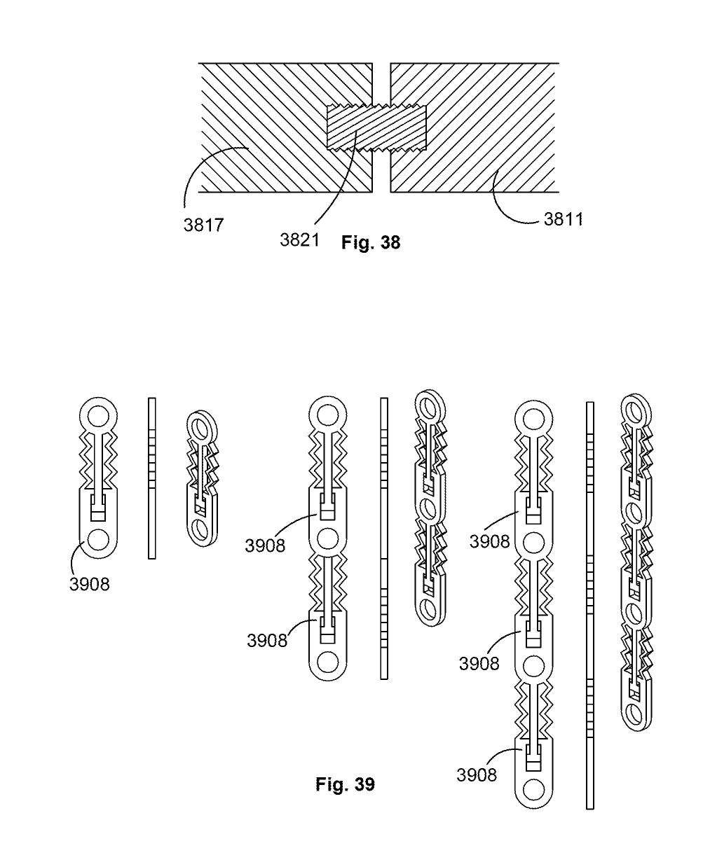

In a further alternative, shown in FIG. 38, a threaded advancement mechanism is used wherein an eccentrically oriented screwdriver can be used to rotate a centrally located thread 3821 that couples first and second spacing portions 3811, 3817 so as to facilitate altering a spacing therebetween.

Often, multiple adjacent intervertebral levels need to be stabilised in a patient. In such cases, multi-level stabilisation can be achieved by either a modular mechanism that allows the introduction or removal of en extra level to a base device, such as the orthopaedic stabilisation devices 100, 400, or by providing multiple orthopaedic stabilisation devices that are capable of stabilising a different number of levels.

Examples of multi-level stabilisation devices are illustrated in FIG. 39. In these examples, a plurality of stabilisation members 3908 are coupled together in series. In this case multiple models of the orthopaedic stabilisation device 100 are manufactured and a surgeon can choose the appropriate model.

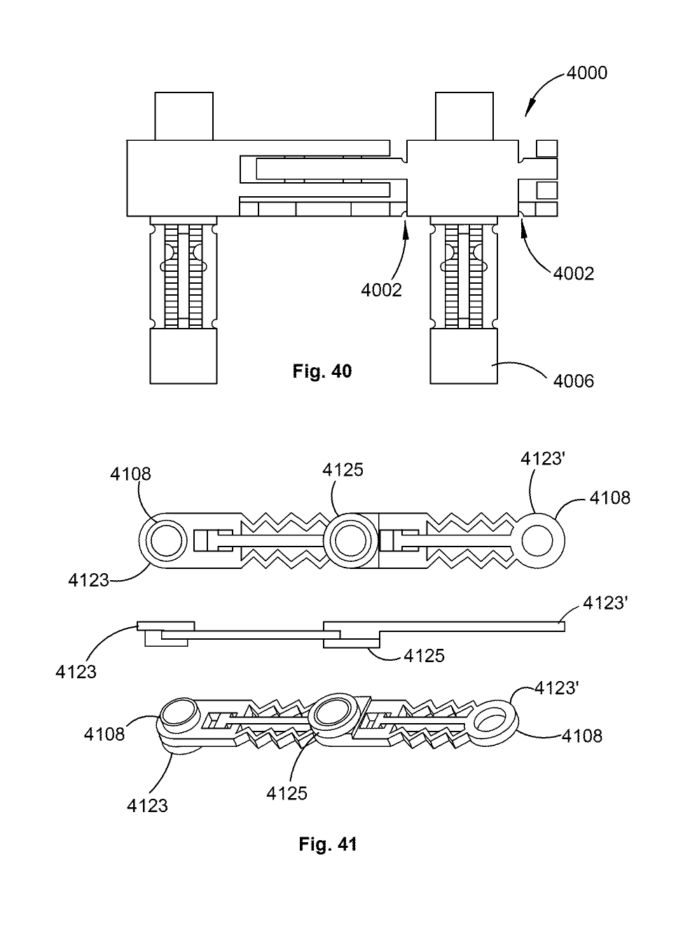

FIG. 40 shows an example wherein a multi-level orthopaedic stabilisation device 4000 comprises notches 4002 arranged adjacent a leg 4006 to allow a surgeon to remove unnecessary levels by cutting the device 4000 at the notches 4002. For example, a three level orthopaedic stabilisation device can be provided and the surgeon can cut the device down to size as required. This can either be performed using standard surgical tools or a specially designed instrument provided with the orthopaedic stabilisation device 100.

Alternatively, and as shown in FIG. 41, modularity of stabilisation members 4108 can be achieved by providing connectable portions 4123, 4123′ that can be coupled together and retained by threaded attachment of a retaining washer 4125.

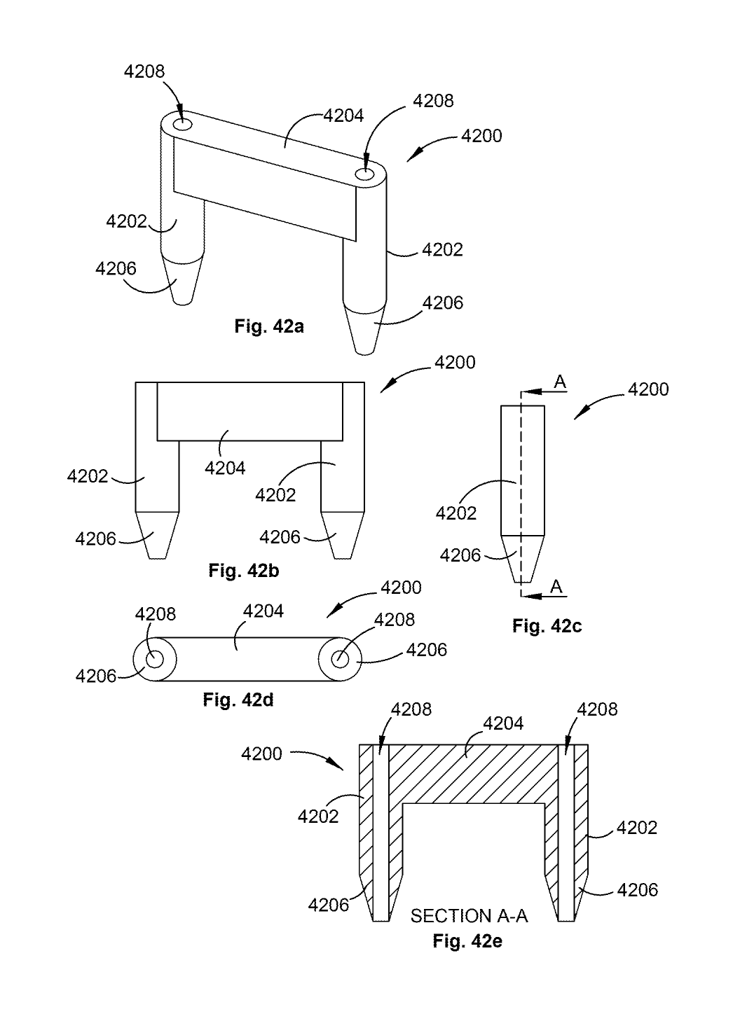

Implantation of the orthopaedic stabilisation device 100 may be assisted by an awling tool 4200 as shown in FIGS. 42a to 42e. The awling tool comprises two legs 4202 that are coupled by bridging member 4204. Each leg has a frustoconical end portion 4206 that is arranged to facilitate positioning the awling tool 4200 against bones or bone portions that are to be stabilised by implantation of the orthopaedic stabilisation device 100.

When positioned against the bones or bone portions, a drill or similar can be inserted through respective passages 4208 of each leg to facilitate forming a bore hole in the bones or bone portions. A distance between the respective passages 4208 corresponds to a distance between central axes of the legs 106 of the orthopaedic stabilisation device 100, and therefore the awling tool 4200 can be used to form bore holes that are appropriately spaced to facilitate implantation of each leg 106 into their respective bore holes.

Numerous variations and modifications will suggest themselves to persons skilled in the relevant art, in addition to those already described, without departing from the basic inventive concepts. All such variations and modifications are to be considered within the scope of the present invention, the nature of which is to be determined from the foregoing description.

In the description of the invention, except where the context requires otherwise due to express language or necessary implication, the words “comprise” or variations such as “comprises” or “comprising” are used in an inclusive sense, i.e. to specify the presence of the stated features, but not to preclude the presence or addition of further features in various embodiments of the invention.

1. A method of inserting an orthopedic stabilization device, the stabilisation device comprising a stabilization member and at least two legs coupled to the stabilization member, the at least two legs being arranged for positioning in a first bore hole in a first bone portion or bone and a second bore hole in a second bone portion or bone, respectively, for receiving an element within each of the first leg and the second leg such that the element can facilitate fastening each of the first leg and the second leg within the respective first and second bone portions or bones, wherein the first leg and the second leg are coupled to the stabilization member prior to positioning the first leg and the second leg in the respective first and second bore holes such that orthopedic stabilization device can be positioned in a stabilizing position as a single unit; and wherein the stabilisation member comprises first and second stabilisation portions that are moveable relative to each other whereby the stabilisation member is arranged such that a length of the stabilisation member is alterable by a predefined amount, the predefined amount being adjustable,
the method comprising:
providing the orthopedic stabilization device with the first and second legs being coupled to the stabilization member and the length of the stabilisation member being adjusted by the predefined amount; thereafter
positioning the first leg and the second leg of the orthopedic stabilization device into the respective first and second bore holes of the respective first and second bone portions or bones in the stabilizing position as a single unit; and
stabilizing the first and second bones with respect to one another using the orthopedic stabilization device.

2. The method of claim 1 comprising coupling the at least two legs to the stabilization member prior to positioning the first leg and the second leg of the orthopedic stabilization device to the respective first and second bore holes of the respective first and second bones in the stabilisation position as a single unit.

3. The orthopedic stabilization device of claim 1, wherein each of the at least two legs has a symmetric axial profile.

4. The method of claim 1, wherein at least one leg is moveable from a contracted configuration to an expanded configuration, the at least one leg being arranged to be received within a respective bore hole when in the contracted configuration and to be moveable to the expanded configuration when located in the respective bore hole for facilitating fastening the at least one leg within the respective bore hole, and wherein the element that results in fastening is an actuating member, the at least one leg being arranged to receive the actuating member such that interaction between the actuating member and the at least one leg facilitates moving the at least one leg from the contracted configuration to the expanded configuration.

5. The method of claim 4, wherein the expanded configuration of the at least one leg is a configuration wherein at least a portion of the at least one leg has an increased radial dimension compared to when the at least one leg is in the contracted configuration.

6. The method of claim 4, wherein the stabilization device comprises the actuating member.

7. The method of claim 6, wherein at least a portion of the actuating member is separable from the actuating member.

8. The method of claim 4, wherein each leg is moveable from a contracted to the expanded configuration, and wherein each leg can be moved from the contracted to the expanded configuration at substantially the same time.

9. The method of claim 8, wherein the actuating member is arranged so as to effect movement of each leg from the contracted to the expanded configuration at substantially the same time.

10. The method of claim 4, wherein an internal wall of the at least one leg is shaped so as to engage with at least a portion of the actuating member to retain the actuating member when the actuating member has caused the at least one leg to move to the expanded configuration.
